(19) United States
(12) Patent Application Publication
(10) Pub. No.: US 2021/0108275 A1
(43) Pub. Date: Apr. 15, 2021

(54) MATERIALS AND METHODS FOR BLADDER CANCER DETECTION

(71) Applicant: STEVEN GOODISON, JACKSONVILLE, FL (US)

(72) Inventor: STEVEN GOODISON, JACKSONVILLE, FL (US)

(21) Appl. No.: 17/131,943

(22) Filed: Dec. 23, 2020

Related U.S. Application Data
(63) Continuation of application No. 15/918,626, filed on Mar. 12, 2018.
(60) Provisional application No. 62/470,282, filed on Mar. 12, 2017.

(51) Int. Cl. C12Q 1/6886

(52) U.S. Cl. CPC C12Q 1/6886; C12Q 2600/178; C12Q 2600/158

(57) ABSTRACT

The subject invention pertains to biomarkers for identifying BC in a subject. The biomarkers presented herein include miRNAs, particularly, a combination of miRNAs. The invention demonstrates that the specific miRNAs are increased body fluid, particularly, urine, from a subject compared to that of a control. Accordingly, the levels of specific mRNAs, in a body fluid, particularly, urine, from a subject is used to diagnose BC in a subject. The invention also provides kits and reagents to conduct assays to quantify biomarkers described herein. The invention further provides methods of treating and/or managing BC in a subject.

### CROSS-REFERENCE TO RELATED APPLICATIONS

This application is a continuation of co-pending U.S. patent application Ser. No. 15/918,626, filed Mar. 12, 2018; which claims the priority benefit of U.S. Provisional Application Ser. No. 62/470,282, filed Mar. 12, 2017, all of which are incorporated herein by reference in their entirety.

The Sequence Listing for this application is labeled “SeqList12Mar18-ST25.txt,” which was created on Mar. 12, 2018, and is 18 KB. The Sequence Listing is incorporated herein by reference in its entirety.

### BACKGROUND OF INVENTION

With an estimated 75,000 new cases each year in the US, bladder cancer (BC) is a major cause of morbidity and mortality. Although not typically life threatening if detected early, more than 70% of patients with BC will have a recurrence during the first two years after diagnosis. This recurrence phenomenon means that patients face a lifetime of surveillance undergoing multiple invasive procedures.

Current guidelines support a diagnostic approach of cystoscopy coupled with voided urine cytology (VUC). Invasive cystoscopy is associated with significant discomfort, possible infection and trauma. VUC is a non-invasive adjunct to cystoscopy; however, the assay has poor sensitivity, especially for low-grade and low-stage tumors.

MicroRNAs (miRNAs) are a class of short, non-coding RNA molecules that modulate protein expression through the perturbation of mRNA translation. Complementary binding of miRNAs to target mRNA transcripts causes suppression of translation through interference of complex formation or mRNA degradation. Each miRNA can have multiple targets; therefore, changes in the profile of expressed miRNAs can have magnified effects on cellular phenotype. Although far from fully characterized, specific microRNAs have been implicated in a number of diseases, including cancers.

A number of tests are available to detect BC-associated urinary biomarkers; however, these tests tend to have poor sensitivity and accuracy. To date, four urine tests have received FDA approval for diagnostic clinical use (BTA-Stat, BTA-Trak, NMP22 POC device, and UroVysion FISH test), and a couple others have approval restricted to post-treatment monitoring. In a meta-analysis of 57 studies, although specificity of the current diagnostic tests was in the range of 74% to 88%, none achieved a sensitivity >69%.

### BRIEF SUMMARY

The subject invention provides materials and methods for identifying bladder cancer (BC) in a subject. The biomarkers provided herein include miRNAs. These biomarkers can be detected in a biological sample such as, for example, a tissue biopsy or a body fluid. The sample may be, for example, urine from a subject.

Accordingly, in one embodiment, levels of certain miRNAs in a urine sample from a subject are used to identify BC in the subject.

In one embodiment, alterations in the levels of specific miRNAs, particularly, a combination of 10 to 25 miRNAs, particularly, the combinations of 10, 15, 20, or 25 miRNAs, in a body fluid sample from a subject, are used to identify BC in the subject.

The invention also provides methods of treating and/or managing BC in a subject by administering a therapy to the subject.

The invention further provides kits and reagents to conduct assays to detect and/or quantify the biomarkers described herein.

### BRIEF DESCRIPTION OF DRAWING

FIG. 1 is a ROC curve illustrating the diagnostic accuracy of 3 miRNA set classifiers for predicting the presence of BC. Curves are presented for an optimal 25-miRNA model and for models restricted to 10, 15 and 20 miRNAs.

### BRIEF DESCRIPTION OF THE SEQUENCES

SEQ ID NOs: 1 to 92: Sequences of pre-miRNAs (stem-loop miRNA) and mature miRNAs that are increased in body fluid samples from BC subjects as indicated below.

| miRNA ID | Mature miRNA Sequence | SEQ ID NO: | Stem-loop Sequence | SEQ ID NO: |
|---|---|---|---|---|
| hsa-miR- | CAGUGGUUUUACCCUAUGGUA | 1 | UGUGUCUCUCUCUGUGUCCUGCCAGUGGUUUU | 2 |
| A14-5p | G | | ACCCUAUGGUAGGUUACGUCAUGCUGUUCUAC | |
| | | | CACAGGGUAGAACCACGGACAGGAUACCGGGG | |
| | | | CACC | |
| hsa-miR- | CAUAAAGUAGAAAGCACUACU | 3 | GACAGUGCAGUCACCCAUAAAGUAGAAAGCAC | 4 |
| 142-5p | | | UACUAACAGCACUGGAGGGUGUAGUGUUUCCU | |
| | | | ACUUUAUGGAUGAGUGUACUGUG | |
| hsa-miR- | ACAGUAGUCUGCACAUUGGUU | 5 | GCCAACCCAGUGUUCAGACUACCUGUUCAGGA | 6 |
| 199a-3p | A | | GGCUCUCAAUGUGUACAGUAGUCUGCACAUUG | |
| | | | GUUAGGC | |
| hsa-miR- | CAAAGUGCUGUUCGUGCAGGU | 7 | CUGGGGGCUCCAAAGUGCUGUUCGUGCAGGUA | 8 |
| 93-5p | AG | | GUGUGAUUACCUGACCUACUGCUGAGCUAGCA | |
| | | | CUUCCCGAGCCCCGG | |
| hsa-miR- | AAUGGCGCCACUAGGGUUGUG | 9 | ACGAAUGGCUAUGCACUGCACAACCCUAGGAG | 10 |
| 652-3p | | | AGGGUGCCAUUCACAUAGACUAUAAUUGAAUG | |
| | | | GCGCCACUAGGGUUGUGCAGUGCACAACCUAC | |
| | | | AC | |
| hsa-miR- | UAAAGUGCUUAUAGUGCAGGU | 11 | GUAGCACUAAAGUGCUUAUAGUGCAGGUAGUG | 12 |
| 20a-5p | AG | | UUUAGUUAUCUACUGCAUUAUGAGCACUUAAA | |
| | | | GUACUGC | |
| hsa-miR- | CCGCACUGUGGGUACUUGCUG | 13 | CCUGCCGGGGCUAAAGUGCUGACAGUGCAGAU | 14 |
| 106b-3p | C | | AGUGGUCCUCUCCGUGCUACCGCACUGUGGGU | |
| | | | ACUUGCUGCUCCAGCAGG | |
| hsa-miR- | UUUUCAACUCUAAUGGGAGAG | 15 | AAGAUCCUGCUGUUUCUACCAUUAGUUUUGAA | 16 |
| 1305 | A | | UGUUUAUUGUAAAGAUACUUUUCAACUCUAAU | |
| | | | GGGAGAGACAGCAGGAUUCUCC | |
| hsa-miR- | UGUCAGUUUGUCAAAUACCCC | 17 | CCUGGCCUCCUGCAGUGCCACGCUCCGUGUAU | 18 |
| 223-3p | A | | UUGACAAGCUGAGUUGGACACUCCAUGUGGUA | |
| | | | GAGUGUCAGUUUGUCAAAUACCCCAAGUGCGG | |
| | | | CACAUGCUUACCAG | |
| hsa-miR- | UAAGGUGCAUCUAGUGCAGAU | 19 | UGUUCUAAGGUGCAUCUAGUGCAGAUAGUGAA | 20 |
| 18a-5p | AG | | GUAGAUUAGCAUCUACUGCCCUAAGUGCUCCU | |
| | | | UCUGGCA | |
| hsa-miR- | CAACGGAAUCCCAAAAGCAGC | 21 | CGGCUGGACAGCGGGCAACGGAAUCCCAAAAG | 22 |
| 191-5p | UG | | CAGCUGUUGUCUCCAGAGCAUUCCAGCUGCGC | |
| | | | UUGGAUUUCGUCCCCUGCUCUCCUGCCU | |
| hsa-miR- | UCGUACCGUGAGUAAUAAUGC | 23 | CGCUGGCGACGGGACAUUAUUACUUUUGGUAC | 24 |
| 126-3p | G | | GCGCUGUGACACUUCAAACUCGUACCGUGAGU | |
| | | | AAUAAUGCGCCGUCCACGGCA | |
| hsa-miR- | UUCAAGUAAUUCAGGAUAGGU | 25 | CCGGGACCCAGUUCAAGUAAUUCAGGAUAGGU | 26 |
| 26b-5p | | | GUGUGCUGUCCAGCCUGUUCUCCAUUACUUGG | |
| | | | CUCGGGGACCGG | |
| hsa-miR- | UUCAAGUAAUCCAGGAUAGGC | 27 | GUGGCCUCGUUCAAGUAAUCCAGGAUAGGCUG | 28 |
| 26a-5p | U | | UGCAGGUCCCAAUGGGGCCUAUUCUUGGUUAC | |
| | | | UUGCACGGGGACGC | |
| hsa-miR- | GUCCAGUUUUCCCAGGAAUCC | 29 | CACCUUGUCCUCACGGUCCAGUUUUCCCAGGA | 30 |
| 145-5p | CU | | AUCCCUUAGAUGCUAAGAUGGGGAUUCCUGGA | |
| | | | AAUACUGUUCUUGAGGUCAUGGUU | |
| hsa-miR- | UGAGAACUGAAUUCCAUGGGU | 31 | CCGAUGUGUAUCCUCAGCUUUGAGAACUGAAU | 32 |
| 146a-5p | U | | UCCAUGGGUUGUGUCAGUGUCAGACCUCUGAA | |
| | | | AUUCAGUUCUUCAGCUGGGAUAUCUCUGUCAU | |
| | | | CGU | |
| hsa-miR- | CUUUCAGUCGGAUGUUUGCAG | 33 | GCGACUGUAAACAUCCUCGACUGGAAGCUGUG | 34 |
| 30a-3p: | C | | AAGCCACAGAUGGGCUUUCAGUCGGAUGUUUG | |
| | | | CAGCUGC | |
| hsa-miR- | UUUGGCACUAGCACAUUUUUG | 35 | UGGCCGAUUUUGGCACUAGCACAUUUUUGCUU | 36 |
| 96-5p | CU | | GUGUCUCUCCGCUCUGAGCAAUCAUGUGCAGU | |
| | | | GCCAAUAUGGGAAA | |
| hsa-miR- | CUGAAGUGAUGUGUAACUGAU | 37 | UUUAGCGGUUUCUCCCUGAAGUGAUGUGUAAC | 38 |
| 573 | CAG | | UGAUCAGGAUCUACUCAUGUCGUCUUUGGUAA | |
| | | | AGUUAUGUCGCUUGUCAGGGUGAGGAGAGUUU | |
| | | | UUG | |
| hsa-miR- | AGCUACAUUGUCUGCUGGGUU | 39 | UGAACAUCCAGGUCUGGGGCAUGAACCUGGCA | 40 |
| 221-3p | UC | | UACAAUGUAGAUUUCUGUGUUCGUUAGGCAAC | |
| | | | AGCUACAUUGUCUGCUGGGUUUCAGGCUACCU | |
| | | | GGAAACAUGUUCUC | |
| hsa-miR- | UUUGGCAAUGGUAGAACUCAC | 41 | GAGCUGCUUGCCUCCCCCCGUUUUUGGCAAUG | 42 |
| 182-5p | ACU | | GUAGAACUCACACUGGUGAGGUAACAGGAUCC | |
| | | | GGUGGUUCUAGACUUGCCAACUAUGGGGCGAG | |
| | | | GACUCAGCCGGCAC | |
| hsa-miR- | UGUAGUGUUUCCUACUUUAUG | 43 | GACAGUGCAGUCACCCAUAAAGUAGAAAGCAC | 44 |
| 142-3p | GA | | UACUAACAGCACUGGAGGGUGUAGUGUUUCCU | |
| | | | ACUUUAUGGAUGAGUGUACUGUG | |
| hsa-miR- | UGUGCAAAUCCAUGCAAAACU | 45 | CACUGUUCUAUGGUUAGUUUUGCAGGUUUGCA | 46 |
| 19b-3p | GA | | UCCAGCUGUGUGAUAUUCUGCUGUGCAAAUCC | |
| | | | AUGCAAAACUGACUGUGGUAGUG | |
| hsa-miR- | CAAGUCACUAGUGGUUCCGUU | 47 | GGGCUUUCAAGUCACUAGUGGUUCCGUUUAGU | 48 |
| 224-5p | | | AGAUGAUUGUGCAUUGUUUCAAAAUGGUGCCC | |
| | | | UAGUGACUACAAAGCCC | |
| hsa-miR- | AACAUUCAACGCUGUCGGUGA | 49 | AGAAGGGCUAUCAGGCCAGCCUUCAGAGGACU | 50 |
| 181a-5p | GU | | CCAAGGAACAUUCAACGCUGUCGGUGAGUUUG | |
| | | | GGAUUUGAAAAAACCACUGACCGUUGACUGUA | |
| | | | CCUUGGGGUCCUUA | |
| hsa-miR- | ACUCCAGCCCCACAGCCUCAG | 51 | GCAUCCUCAGGACCUGGGCUUGGGUGGUAGGA | 52 |
| 766-3p | C | | GGAAUUGGUGCUGGUCUUUCAUUUUGGAUUUG | |
| | | | ACUCCAGCCCCACAGCCUCAGCCACCCCAGCC | |
| | | | AAUUGUCAUAGGAGC | |
| hsa-miR- | UGAGAACUGAAUUCCAUAGGC | 53 | CCUGGCACUGAGAACUGAAUUCCAUAGGCUGU | 54 |
| 146b-5p | U | | GAGCUCUAGCAAUGCCCUGUGGACUCAGUUCU | |
| | | | GGUGCCCGG | |
| hsa-miR- | UAAUACUGUCUGGUAAAACCG | 55 | UGCCGGCCGAUGGGCGUCUUACCAGACAUGGU | 56 |
| 429 | U | | UAGACCUGGCCCUCUGUCUAAUACUGUCUGGU | |
| | | | AAAACCGUCCAUCCGCUGC | |
| hsa-miR- | UAACACUGUCUGGUAACGAUG | 57 | CCGGGCCCCUGUGAGCAUCUUACCGGACAGUG | 58 |
| 200a-3p | U | | CUGGAUUUCCCAGCUUGACUCUAACACUGUCU | |
| | | | GGUAACGAUGUUCAAAGGUGACCCGC | |
| hsa-miR- | UAAUACUGCCGGGUAAUGAUG | 59 | CCCUCGUCUUACCCAGCAGUGUUUGGGUGCGG | 60 |
| 200c-3p | GA | | UUGGGAGUCUCUAAUACUGCCGGGUAAUGAUG | |
| | | | GAGG | |
| hsa-miR- | CAAAGUGCUCAUAGUGCAGGU | 61 | AGUACCAAAGUGCUCAUAGUGCAGGUAGUUUU | 62 |
| 20b-5p | AG | | GGCAUGACUCUACUGUAGUAUGGGCACUUCCA | |
| | | | GUACU | |
| hsa-miR- | ACUGCCCCAGGUGCUGCUGG | 63 | CUGACUAUGCCUCCCCGCAUCCCCUAGGGCAU | 64 |
| 324-3p | | | UGGUGUAAAGCUGGAGACCCACUGCCCCAGGU | |
| | | | GCUGCUGGGGGUUGUAGUC | |
| hsa-miR- | UGUGCAAAUCUAUGCAAAACU | 65 | GCAGUCCUCUGUUAGUUUUGCAUAGUUGCACU | 66 |
| 19a-3p | GA | | ACAAGAAGAAUGUAGUUGUGCAAAUCUAUGCA | |
| | | | AAACUGAUGGUGGCCUGC | |
| hsa-miR- | AAAAGUGCUUACAGUGCAGGU | 67 | CCUUGGCCAUGUAAAAGUGCUUACAGUGCAGG | 68 |
| 106a-5p | AG | | UAGCUUUUUGAGAUCUACUGCAAUGUAAGCAC | |
| | | | UUCUUACAUUACCAUGG | |
| hsa-miR- | UGAGAUGAAGCACUGUAGCUC | 69 | GCGCAGCGCCCUGUCUCCCAGCCUGAGGUGCA | 70 |
| 143-3p | | | GUGCUGCAUCUCUGGUCAGUUGGGAGUCUGAG | |
| | | | AUGAAGCACUGUAGCUCAGGAAGAGAGAAGUU | |
| | | | GUUCUGCAGC | |
| hsa-miR- | CACCCGUAGAACCGACCUUGC | 71 | GGCACCCACCCGUAGAACCGACCUUGCGGGGC | 72 |
| 99b-5p | G | | CUUCGCCGCACACAAGCUCGUGUCUGUGGGUC | |
| | | | CGUGUC | |
| hsa-miR- | UACCACAGGGUAGAACCACGG | 73 | UGUGUCUCUCUCUGUGUCCUGCCAGUGGUUUU | 74 |
| 140-3p | | | ACCCUAUGGUAGGUUACGUCAUGCUGUUCUAC | |
| | | | CACAGGGUAGAACCACGGACAGGAUACCGGGG | |
| | | | CACC | |
| hsa-miR- | AGUGGGGAACCCUUCCAUGAG | 75 | UUGACUUAGCUGGGUAGUGGGGAACCCUUCCA | 76 |
| 491-5p | G | | UGAGGAGUAGAACACUCCUUAUGCAAGAUUCC | |
| | | | CUUCUACCUGGCUGGGUUGG | |
| hsa-miR- | CUAGACUGAAGCUCCUUGAGG | 77 | UUUCCUGCCCUCGAGGAGCUCACAGUCUAGUA | 78 |
| 151a-3p | | | UGUCUCAUCCCUACUAGACUGAAGCUCCUUGA | |
| | | | GGACAGGGAUGGUCAUACUCACCUC | |
| hsa-miR- | UCCGGUUCUCAGGGCUCCACC | 79 | GCAGGUGAACUGGCAGGCCAGGAAGAGGAGGA | 80 |
| 671-3p | | | AGCCCUGGAGGGGCUGGAGGUGAUGGAUGUUU | |
| | | | UCCUCCGGUUCUCAGGGCUCCACCUCUUUCGG | |
| | | | GCCGUAGAGCCAGGGCUGGUGC | |
| hsa-miR- | AGCUACAUCUGGCUACUGGGU | 81 | GCUGCUGGAAGGUGUAGGUACCCUCAAUGGCU | 82 |
| 222-3p | | | CAGUAGCCAGUGUAGAUCCUGUCUUUCGUAAU | |
| | | | CAGCAGCUACAUCUGGCUACUGGGUCUCUGAU | |
| | | | GGCAUCUUCUAGCU | |
| hsa-miR- | UGAGCGCCUCGACGACAGAGC | 83 | CGGGGCGGCCGCUCUCCCUGUCCUCCAGGAGC | 84 |
| 339-3p | CG | | UCACGUGUGCCUGCCUGUGAGCGCCUCGACGA | |
| | | | CAGAGCCGGCGCCUGCCCCAGUGUCUGCGC | |
| hsa-miR- | UAACACUGUCUGGUAAAGAUG | 85 | CGGCCGGCCCUGGGUCCAUCUUCCAGUACAGU | 86 |
| 141-3p | G | | GUUGGAUGGUCUAAUUGUGAAGCUCCUAACAC | |
| | | | UGUCUGGUAAAGAUGGCUCCCGGGUGGGUUC | |
| hsa-miR- | UAAUACUGCCUGGUAAUGAUG | 87 | CCAGCUCGGGCAGCCGUGGCCAUCUUACUGGG | 88 |
| 200b-3p | A | | CAGCAUUGGAUGGAGUCAGGUCUCUAAUACUG | |
| | | | CCUGGUAAUGAUGACGGCGGAGCCCUGCACG | |
| hsa-let- | UGAGGUAGUAGGUUGUGUGGU | 89 | CGGGGUGAGGUAGUAGGUUGUGUGGUUUCAGG | 90 |
| 7b-5p | U | | GCAGUGAUGUUGCCCCUCGGAAGAUAACUAUA | |
| | | | CAACCUACUGCCUUCCCUG | |
| hsa-miR- | UAGCUUAUCAGACUGAUGUUG | 91 | UGUCGGGUAGCUUAUCAGACUGAUGUUGACUG | 92 |
| 21-5p | A | | UUGAAUCUCAUGGCAACACCAGUCGAUGGGCU | |
| | | | GUCUGACA | |

### DETAILED DISCLOSURE

In accordance with the subject invention one or more miRNAs, preferably panels of multiple miRNA biomarkers, are used in a non-invasive assay for the detection of BC in a subject.

From 754 human miRNAs in a cohort of 85 subjects, 25 miRNAs were identified as being significantly associated with the presence of BC and these were monitored in an independent validation cohort of 121 subjects using quantitative real-time PCR (RT-PCR). A significant association with the presence of BC was confirmed for certain miRNA biomarkers in the validation study. Further, multivariate modeling identified 10 to 25 target biomarker signatures that achieved an advantageous diagnostic performance, namely, AUROC of 1.0 and sensitivity and specificity of about 100%.

In multivariate analyses, a 25-miRNA panel provided herein correctly predicted the disease status of 121 subjects (61 with BC). Restriction to 20, 15, and 10 miRNA prediction models resulted in a reduction in performance values; however, the smaller miRNA models achieved sensitivity and specificity above 84% (AUC>0.9). These values are comparable to, or better than, the performance of cystoscopy and VUC.

Several miRNAs (hsa-miR-140-5p, hsa-miR-199a-3p, hsa-miR-93-5p, hsa-miR-652-3p, hsa-miR-1305, hsa-miR-766-3p, and hsa-miR-96-5p) consistently contributed to all models.

Accordingly, certain embodiments of the invention provide panels of miRNA biomarkers that can detect BC in a subject based on the analysis of a sample, particularly, a sample obtained in a non-invasive manner, such as urine.

In specific embodiments, the claimed invention provides panels of 25 miRNAs (identified in Table 2), between 10 to 25 miRNAs, particularly, 10, 15, 20 or 25 miRNAs, wherein each miRNA in the panel of miRNAs is associated with BC in a subject and the panel of miRNAs, when analyzed as a combination, can be used to diagnose and/or monitor BC in the subject. Certain embodiments of the invention provide a panel of 10 miRNAs, 15 miRNAs, 20 miRNAs or 25 miRNAs, indicated below:

A panel of 10 miRNAs comprises, consists of, or consists essentially of: hsa-miR-652-3p, hsa-miR-199a-3p, hsa-miR-140-5p, hsa-miR-93-5p, hsa-miR-142-5p, hsa-miR-1305, hsa-miR-30a-3p, hsa-miR-224-5p, hsa-miR-96-5p, and hsa-miR-766-3p. This panel is hereinafter referred to as “Panel 10.”

A panel of 15 miRNAs comprises, consists of, or consists essentially of: hsa-miR-652-3p, hsa-miR-199a-3p, hsa-miR-140-5p, hsa-miR-93-5p, hsa-miR-142-5p, hsa-miR-1305 hsa-miR-30a-3p, hsa-miR-224-5p, hsa-miR-96-5p, hsa-miR-766-3p, hsa-miR-223-3p, hsa-miR-99b-5p, hsa-miR-140-3p, hsa-let-7b-5p, and hsa-miR-141-3p. This panel is hereinafter referred to as “Panel 15.”

A panel of 20 miRNAs comprises, consists of, or consists essentially of: hsa-miR-652-3p, hsa-miR-199a-3p, hsa-miR-140-5p, hsa-miR-93-5p, hsa-miR-142-5p, hsa-miR-1305, hsa-miR-30a-3p, hsa-miR-224-5p, hsa-miR-96-5p, hsa-miR-766-3p, hsa-miR-223-3p, hsa-miR-99b-5p, hsa-miR-140-3p, hsa-let-7b-5p, hsa-miR-141-3p, hsa-miR-191-5p, hsa-miR-146b-5p, hsa-miR-491-5p, hsa-miR-339-3p, and hsa-miR-200c-3p. This panel is hereinafter referred to as “Panel 20.”

A panel of 25 miRNAs comprises, consists of, or consists essentially of: hsa-miR-652-3p, hsa-miR-199a-3p, hsa-miR-140-5p, hsa-miR-93-5p, hsa-miR-142-5p, hsa-miR-1305, hsa-miR-30a-3p, hsa-miR-224-5p, hsa-miR-96-5p, hsa-miR-766-3p, hsa-miR-223-3p, hsa-miR-99b-5p, hsa-miR-140-3p, hsa-let-7b-5p, hsa-miR-141-3p, hsa-miR-191-5p, hsa-miR-146b-5p, hsa-miR-491-5p, hsa-miR-339-3p, hsa-miR-200c-3p, hsa-miR-106b-3p, hsa-miR-143-3p, hsa-miR-429, hsa-miR-222-3p, and hsa-miR-200a. This panel is hereinafter referred to as “Panel 25.”

In further embodiments, the claimed invention provides a panel of at least 4, 5, 6, 7, 8, or 9 of the panel of 25 miRNAs. When analyzed as a combination, a panel of at least 4, 5, 6, 7, 8, or 9 of the panel of 25 miRNAs can be used to diagnose BC in a subject with specificity of at least 80%, at least 85%, at least 90%, or at least 95% and sensitivity of at least 80%, at least 85%, at least 90%, or at least 95%. In certain embodiments, the panel of at least 4, 5, 6, 7, 8, or 9 miRNAs is selected from Panel 10, Panel 15, Panel 20, or Panel 25, wherein the panel of miRNAs can be used to diagnose BC in a subject with specificity of at least 80%, at least 85%, at least 90%, or at least 95% and sensitivity of at least 80%, at least 85%, at least 90%, or at least 95%.

In one embodiment, the sensitivity and specificity values are for cohort 1 and/or cohort 2 of Table 1 or another cohort having substantially the same characteristics.

The miRNAs identified in these panels are upregulated or downregulated in BC as indicated in Table 2.

For the purpose of this invention, the term “an miRNA is associated with BC in a subject” indicates that the miRNA is differentially present, i.e., present at a higher or lower level compared to a healthy control, in a tissue or body fluid, particularly, urine, of the subject. The miRNAs in a panel, particularly, when analyzed as a combination, can be used to diagnose, prognose, or monitor BC in a subject.

Accordingly, one embodiment of the invention provides a method of identifying BC in a subject, the method comprising:
(a) determining the level of two or more miRNAs in:
   i) a test sample obtained from the subject, and
   ii) optionally, a control sample;
(b) optionally, obtaining two or more reference values corresponding to levels of two or more miRNAs; and
(c) identifying BC in a subject based on the levels of two or more miRNAs in the test sample and optionally, administering a therapy to the subject to treat and/or manage BC, or
(d) identifying an absence of BC in the subject based on the levels of two or more miRNAs in the test sample and withholding the therapy to the subject to treat and/or manage BC.

Various techniques are well known to a person of ordinary skill in the art to determine the level of miRNA in a sample. Non-limiting examples of such techniques include microarray analysis, real-time polymerase chain reaction (PCR), Northern blot, in situ hybridization, solution hybridization, and quantitative reverse transcription PCR (qRT-PCR). Methods for carrying out these techniques are routine in the art. Additional methods of determining the level of miRNA in a sample are also well known to a person of ordinary skill in the art and such embodiments are within the purview of the invention.

The reference values corresponding to levels of two or more miRNAs indicate the level of miRNA in a tissue or body fluid obtained from subjects that do not have BC or from subjects that are known to have BC. As such, the reference values corresponding to levels of two or more miRNAs may indicate the absence or presence of BC. A reference value associated with the absence of BC may be determined based on samples obtained from subjects known to be free of BC. A reference value associated with the presence of BC may be obtained based on samples obtained from subjects known to have BC.

For example, body fluids from a group of healthy individuals can be obtained and the levels of two or more miRNAs can be determined. The group of subjects can then be monitored for the development of BC. Reference values corresponding to levels of two or more miRNAs that are associated with low risk or no risk of the development of BC or high risk for the development of BC can be determined based on the presence or absence of BC in various subjects whose samples were analyzed. Additional examples of determining reference values associated with no risk or low risk or high risk of the development of BC are well known to a person of ordinary skill in the art and such embodiments are within the purview of the invention.

The step of identifying the subject as having BC utilizes the level of two or more miRNAs in the test sample. For example, if the levels of certain miRNAs in the test sample are significant higher or lower than the levels of corresponding miRNAs in the control sample or the reference values, the subject is identified as having BC. For example, if the levels of two or more miRNAs from Panel 10, Panel 15, Panel 20, or Panel 25 are higher or lower (Table 2) in the test sample compared to control sample or the reference values, the subject is identified as having BC.

In one embodiment, the step of identifying the subject as having BC utilizes the levels of all miRNAs in Panel 10, Panel 15, Panel 20, and/or Panel 25. For example, if the levels of miRNAs from Panel 10, Panel 15, Panel 20, and/or Panel 25 in the test sample are significantly higher or lower (Table 2) than the levels of corresponding miRNAs in the control sample or the reference values, the subject is identified as having BC. Thus, a subject is identified as having BC if the levels of miRNAs from Panel 10, Panel 15, Panel 20, and/or Panel 25 are different in a biological sample from a subject compared to a control sample or the reference values.

In a further embodiment, the step of identifying the subject as having BC utilizes the combined levels of all miRNAs in Panel 10, Panel 15, Panel 20, and/or Panel 25. For example, if the levels of miRNAs from Panel 10, Panel 15, Panel 20, and/or Panel 25 in the test sample are significantly different (Table 2) as a combination, for example, as analyzed by multivariate analysis, than the levels of corresponding miRNAs as a combination in the control sample or the reference values, the subject is identified as having BC.

One embodiment of the invention provides a kit comprising reagents to carry out the methods of the current invention. In one embodiment, the kit comprises primers and/or probes specific for miRNAs. Reagents for treating the samples, for example, deproteination, degradation of DNA, or removal of other impurities, purification of miRNAs, can also be provided in the kit.

One embodiment of the invention provides a kit, for example, a point-of-care (POC) diagnostic device, for assaying two or more miRNAs that can be used to identify the subject as having BC. In another embodiment, the kit comprises an oligonucleotide chip and reagents to conduct the assay to determine the levels of miRNAs corresponding to the oligonucleotides on the oligonucleotide chip. The oligonucleotide chip according to the invention contains oligonucleotides corresponding to a group of miRNAs that are present at different levels in a sample, of a subject having a high risk of the development of BC as compared to the corresponding sample of a subject free from BC.

In one embodiment, the oligonucleotide chip comprises, consists of, or consists essentially of oligonucleotides corresponding to two or more miRNAs selected from Panel 10, Panel 15, Panel 20, or Panel 25 and optionally, two or more control oligonucleotides. In another embodiment, the oligonucleotide chip comprises, consists of, or consists essentially of oligonucleotides corresponding to all the miRNAs from Panel 10, Panel 15, Panel 20, or Panel 25 and optionally, two or more control oligonucleotides.

For the purposes of the invention, the term “oligonucleotide chip consists essentially of oligonucleotides” means that the oligonucleotide chip contains oligonucleotides corresponding to only those miRNAs that present at different levels, either alone or as a combination, in a sample of a subject having BC as compared to the corresponding sample of a subject free from BC and optionally, contains one or more control oligonucleotides.

Also, for the purpose of this invention, the term “a combination consisting essentially of certain miRNAs” means that the combination contains only those certain miRNAs and optionally, one or more control oligonucleotides that are present at levels that are not different, either alone or as a combination, in a sample of a subject having BC as compared to the corresponding sample of a subject free from BC. Further, a kit comprising “a combination consisting essentially of certain miRNAs” indicates that the kit only contains specific miRNAs and can further comprise components other than miRNAs, such as buffers and reagents.

The control oligonucleotides are oligonucleotides corresponding to an miRNA or messenger RNAs (mRNA) known to be present in an equal amount in a sample of a subject having BC as compared to the corresponding sample of a subject free from BC. Non-limiting examples of control oligonucleotides include oligonucleotides corresponding to mRNAs of 18S, U6 form microRNA, β-actin, β-glucoronidase and Glyceraldehyde-3-phosphate dehydrogenase (GAPDH). Additional examples of control miRNAs or mRNAs depend on the sample under examination. A person of ordinary skill in the art can determine control oligonucleotides appropriate for a particular assay and such embodiments are within the purview of the invention.

To practice the methods described herein for identifying a subject as having BC, control samples can be obtained from one or more of the following:
a) an individual belonging to the same species as the subject and not having BC,
b) an individual belonging to the same species as the subject and known to have a low risk of developing BC, or
c) the subject prior to having BC.

Additional examples of control samples are well known to a person of ordinary skill in the art and such embodiments are within the purview of the current invention.

In certain embodiments, the control sample and the test sample are obtained from the same type of an organ or tissue. Non-limiting examples of the organ or tissue that can be used as samples are placenta, brain, eyes, pineal gland, pituitary gland, thyroid gland, parathyroid glands, thorax, heart, lung, esophagus, thymus gland, pleura, adrenal glands, appendix, gall bladder, urinary bladder, large intestine, small intestine, kidneys, liver, pancreas, spleen, stoma, ovaries, uterus, skin, blood or buffy coat sample of blood. Additional examples of organs and tissues are well known to a person of ordinary skill in the art and such embodiments are within the purview of the invention.

In certain other embodiments, the control sample and the test sample are obtained from the same type of a body fluid. Non-limiting examples of the body fluids that can be used as samples include, urine, aqueous humor, vitreous humor, bile, blood, cerebrospinal fluid, chyle, endolymph, perilymph, lymph, mucus (including nasal drainage and phlegm), pericardial fluid, peritoneal fluid, pleural fluid, pus, rheum, saliva, sputum, synovial fluid, vaginal secretion, blood, serum or plasma. Additional examples of body fluids are well known to a person of ordinary skill in the art and such embodiments are within the purview of the invention.

In certain embodiments, the subject is a mammal. Non-limiting examples of mammals include human, ape, canine, pig, bovine, rodent, or feline.

In certain embodiments, the methods of detecting BC in a subject are performed in combination with other tests that are used for detecting BC in a subject. The combination of two or more tests can provide higher sensitivity and specificity.

Once a subject is identified as having BC based on the methods described herein, the step of treating and/or managing BC includes, for example, one, two, three or more of:
i) surgery such as, for example, transurethral resection of bladder tumor (TURBT), or partial or radical cystectomy,
ii) intravesical immunotherapy or intravesical chemotherapy where a drug is administered into the bladder such as, for example, through a catheter rather than by mount or a systemic injection; drugs include Mitomycin, valrubicin, docetaxel, thiotepa, and gemcitabine,
iii) chemotherapy in combination with radiotherapy, for example, cisplatin, cisplatin plus fluorouracil, or mitomycin in combination with radiotherapy,
iv) chemotherapy without radiotherapy, for example, combinations of Gemcitabine and cisplatin; Methotrexate, vinblastine, doxorubicin, and cisplatin; Cisplatin, methotrexate, and vinblastine (called CMV); or Carboplatin and either paclitaxel or docetaxel (for patients with poor kidney function);
v) radiation alone or in combination with surgery or chemotherapy; and
vi) immunotherapy, for example, intravesical BCG, immune checkpoint inhibitors, for example, PD-L1 blockers such as Atezolizumab.

miRNAs that are overexpressed in BC can be oncogenic miRNAs, i.e., miRNAs the overexpression of which contributes to the development of BC. Also, the miRNAs that are reduced in BC can be tumor suppressor miRNAs, i.e., the expression of which contributes to preventing the development of BC. Administering antisense miRNAs that target the miRNAs that are overexpressed in BC can degrade the miRNAs that can be oncogenic. Administering the miRNAs that are reduced in BC can provide tumor suppressing function in a subject having BC.

Therefore, in a particular embodiment, the step of treating and/or managing BC comprises administering to the subject a pharmaceutically effective amount of a combination of:
i) one or more antisense miRNAs that target one or more miRNAs that are upregulated in BC, and/or
ii) one or more miRNAs that are downregulated in BC.

For example, the step of treating and/or managing BC can comprise administering to the subject a pharmaceutically effective amount of:
a) one or more antisense miRNAs that target one or more miRNAs from Panel 10, Panel 15, Panel 20, or Panel 25 that are upregulated in BC; and/or
b) one or more miRNAs that are downregulated in BC.

In a particular embodiment, the antisense miRNAs and/or miRNAs are administered to a subject in a manner that specifically targets the antisense miRNAs and/or miRNAs to BC in the subject. Methods of specifically targeting an agent to certain cells in a subject are well known in the art and such embodiments are within the purview of the invention.

Additional examples of treatments of BC are known in the art and such embodiments are within the purview of the invention.

A further embodiment of the invention provides a method for monitoring the effect of a treatment for BC in a subject. A method for monitoring the effect of a treatment for BC in a subject can comprise:
(a) determining the level of two or more miRNAs in:
   i) a pre-treatment test sample obtained from the subject before the treatment,
   ii) a post-treatment test sample obtained from the subject after the treatment, and
   ii) optionally, a control sample;
(b) optionally obtaining two or more reference values corresponding to levels of two or more miRNAs; and
(c) identifying the treatment for BC in the subject as effective based on the levels of two or more miRNAs in the post-treatment test sample compared to the levels of two or more miRNAs in the pre-treatment test sample and optionally, continuing the treatment for BC in the subject, or
(d) identifying the treatment for BC in the subject as ineffective based on the levels of two or more miRNAs in the post-treatment test sample compared to the levels of two or more miRNAs in the pre-treatment test sample and optionally, modifying the treatment for BC in the subject.

The techniques for determining the levels of miRNAs in a sample, the control samples, and the reference values discussed above in connection with the methods for identifying BC in a subject are also applicable to the methods of monitoring the effect of a treatment for BC described herein.

The step of identifying the treatment for BC in the subject as ineffective utilizes the level of two or more miRNAs in the post-treatment test sample and pre-treatment test sample. An effective treatment is indicated by a decrease in the level of an miRNA in a post-treatment test sample when compared to the pre-treatment test sample, wherein the miRNA is increased in BC. Similarly, an effective treatment is indicated by an increase in the level of an miRNA in a post-treatment test sample when compared to the pre-treatment test sample, wherein the miRNA is decreased in BC. As such, an effective treatment reverts the levels of one or more miRNAs in a subject having BC towards the level that is found in a subject who does not have BC. For example, if the levels of two or more miRNAs from Panel 10, Panel 15, Panel 20, and/or Panel 25 are significantly higher or lower in the post-treatment test sample compared to the pre-treatment test sample, the treatment for BC in the subject is effective. Similarly, if the levels of miRNAs from Panel 10, Panel 15, Panel 20, and/or Panel 25 are significantly higher or lower in the post-treatment test sample compared to the pre-treatment test sample, the treatment for BC in the subject is effective.

Conversely, an ineffective treatment is indicated by no change in the levels of one or more miRNAs in a post-treatment test sample when compared to the pre-treatment test sample, wherein the miRNA is increased or decreased in BC. For example, if the levels of two or more miRNAs from Panel 10, Panel 15, Panel 20, and/or Panel 25 are not significantly different in the post-treatment test sample compared to the pre-treatment test sample, the treatment for BC in the subject is ineffective. Similarly, if the levels of miRNAs from Panel 10, Panel 15, Panel 20, and/or Panel 25 are not significantly different in the post-treatment test sample compared to the pre-treatment test sample, the treatment for BC in the subject is ineffective.

In certain embodiments, the methods of monitoring the treatment of BC in a subject can be performed in combination with other tests that used for monitoring a treatment of BC in a subject as effective or not effective. The combination of two or more tests can provide higher sensitivity and specificity.

As such, the invention provides that miRNA regulation provides manifestation of effective or ineffective treatment of BC.

An even further embodiment of the invention provides a method for determining the prognosis of BC in a subject. Preferably, for the practice of this embodiment, the sample is tissue, such as excised tissue or a body fluid, such as urine. A method for determining the prognosis of BC in a subject can comprise:
(a) determining the level of two or more miRNAs in:
   i) a pre-treatment test sample obtained from the subject before the treatment,
   ii) a post-treatment test sample obtained from the subject after the treatment, and
   ii) optionally, a control sample;
(b) optionally obtaining two or more reference values corresponding to levels of two or more miRNAs; and
(c) identifying the recurrence for BC in the subject based on the levels of two or more miRNAs in the post-treatment test sample compared to the levels of two or more miRNAs in the pre-treatment test sample and optionally, continuing the treatment for BC in the subject, or
(d) identifying the absence of recurrence of BC in the subject based on the levels of two or more miRNAs in the post-treatment test sample compared to the levels of two or more miRNAs in the pre-treatment test sample and optionally, modifying the treatment for BC in the subject.

The techniques for determining the levels of miRNAs in a sample, the control samples, and the reference values discussed above in connection with the methods for identifying BC in a subject are also applicable to the methods of determining the prognosis of BC described herein.

The step of identifying the recurrence of BC in the subject utilizes the level of two or more miRNAs in the post-treatment test sample and pre-treatment test sample. An absence of recurrence of BC is indicated by a decrease in the level of an miRNA in a post-treatment test sample when compared to the pre-treatment test sample, wherein the miRNA is increased in BC. Similarly, an absence of recurrence of BC is indicated by an increase in the level of an miRNA in a post-treatment test sample when compared to the pre-treatment test sample, wherein the miRNA is decreased in BC. As such, reversion in the levels of one or more miRNAs in a subject having BC to the level that is found in a subject who does not have BC indicates the absence of recurrence of BC in the subject. For example, if the levels of two or more miRNAs from Panel 10, Panel 15, Panel 20, and/or Panel 25 are significantly higher or lower in the post-treatment test sample compared to the pre-treatment test sample, the subject does not have a recurrence of BC.

Conversely, a recurrence of BC in a subject is indicated by no change in the levels of one or more miRNAs in a post-treatment test sample when compared to the pre-treatment test sample, wherein the miRNA is increased or decreased in BC. For example, if the levels of two or more miRNAs from Panel 10, Panel 15, Panel 20, and/or Panel 25 are not significantly different in the post-treatment test sample compared to the pre-treatment test sample, the subject is suffering from a recurrence of BC. Similarly, if the levels of miRNAs from Panel 10, Panel 15, Panel 20, and/or Panel 25 are not significantly different in the post-treatment test sample compared to the pre-treatment test sample, the subject is suffering from the recurrence of BC.

In certain embodiments, the methods of detecting recurrence of BC in a subject are performed in combination with other tests that used for detecting recurrence of BC in a subject. The combination of two or more tests can provide higher sensitivity and specificity.

As such, the invention provides that miRNA regulation provides manifestation of recurrence or absence of recurrence of BC in a subject.

A further embodiment of the invention provides an assay for determining the level of two or more miRNAs in:
i) a test sample obtained from the subject, and
ii) optionally, a control sample;

wherein the two or more miRNAs are selected from Panel 10, Panel 15, Panel 20, or Panel 25.

In one embodiment, the invention provides an assay for determining the level of miRNAs identified in Panel 10, Panel 15, Panel 20, or Panel 25, in:
i) a test sample obtained from the subject, and
ii) optionally, a control sample;

Various techniques are well known to a person of ordinary skill in the art to determine the level of an miRNA in a sample. Non-limiting examples of such techniques include microarray analysis, real-time polymerase chain reaction (PCR), Northern blot, in situ hybridization, solution hybridization, or quantitative reverse transcription PCR (qRT-PCR). Methods for carrying out these techniques are routine in the art. Additional methods of determining the level of miRNA in a sample are also well known to a person of ordinary skill in the art and such embodiments are within the purview of the invention.

For example, a hybridization assay can comprise:
a) obtaining a urine sample from a subject and optionally, a control sample,
b) contacting the urine sample with a panel of oligonucleotides, wherein said panel comprises oligonucleotides, wherein each oligonucleotide from the panel of oligonucleotides hybridizes with one or more miRNA selected from the miRNAs present in Panel 10, Panel 15, Panel 20, and Panel 25, and
c) detecting the presence and quantity of the oligonucleotide-miRNA complexes that form in the sample.

A quantitative PCR assay can comprise:
a) obtaining a urine sample from a subject and optionally, a control sample,
b) contacting the urine sample with a combination of primer pairs and probes and conducting a PCR, wherein each combination of a primer pair and a probe from said combinations of primer pairs and probes amplifies and detects one miRNA selected from group consisting of one or more miRNA selected from the miRNAs present in Panel 10, Panel 15, Panel 20, and Panel 25, and
c) detecting the presence and quantity of the two or more miRNAs in the sample based on the qPCR.

The sequences of the miRNAs described herein are known in the art and a skilled artisan can design a primer pair and a probe for the detection of an miRNA. Such embodiments are within the purview of the invention. For example, the sequences of the miRNAs can be obtained from miRBase: the microRNA database, Version 21, July 2014, available at world-wide-website: mirbase.org/index.shtml

Additional data sources for the sequences of miRNAs include the following references and a skilled artisan can obtain the relevant information from these and other sources available in the art:
miRBase: annotating high confidence microRNAs using deep sequencing data, Kozomara A, Griffiths-Jones S., Nucl Acids Res (2013) 42 (D1): D68-D73;
miRBase: integrating microRNA annotation and deep-sequencing data, Kozomara A, Griffiths-Jones S., Nucl. Acids Res. (2011) 39 (suppl 1): D152-D157;
miRBase: tools for microRNA genomics, Griffiths-Jones S, Saini H K, van Dongen S, Enright A J., Nucl. Acids Res. (2008) 36: D154-D158;
miRBase: microRNA sequences, targets and gene nomenclature, Griffiths-Jones S, Grocock R J, van Dongen S, Bateman A, Enright A J., Nucl. Acids Res. (2006) 34: D140-D144; and
The microRNA Registry, Griffiths-Jones S., Nucl. Acids Res. (2004) 32: D109-D111.

In certain embodiments, primer pairs and probes for particular miRNAs are as provided below:

A further embodiment of the invention provides a method for determining whether the levels of two or more miRNAs are above or below a reference value. An assay for determining whether the levels of two or more miRNAs are above or below a reference value can comprise the steps of:
a) obtaining a test sample from the subject,
b) optionally, obtaining a control sample;
c) conducting an assay to determine the levels of two or more miRNAs in the test sample and if obtained, the control sample,
wherein the two or more miRNAs are selected from the miRNAs present in Panel 10, Panel 15, Panel 20, and/or Panel 25.

The reference values corresponding to levels of two or more miRNAs indicate the level of miRNA in body fluids obtained from subjects that do not have BC or from subjects that are known to have BC. As such, the reference values corresponding to levels of two or more miRNAs may indicate the absence or presence of BC. A reference value associated with the absence of BC may be determined based on samples obtained from subjects known to be free of BC. A reference value associated with the development of BC may be obtained based on samples obtained from subjects known to have BC.

For example, body fluids from a group of healthy individuals can be obtained and the levels of two or more miRNAs can be determined. The group of subjects can then be monitored for the development of BC. Reference values corresponding to levels of two or more miRNAs that are associated with low risk or no risk of the development of BC or high risk for the development of BC can be determined based on the presence or absence of BC in various subjects whose samples were analyzed. Additional examples of determining reference values associated with no risk or low risk or high risk of the development of BC are well known to a person of ordinary skill in the art and such embodiments are within the purview of the invention.

As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” “comprise,” “consisting essentially of,” “consists essentially of,” “consisting” and “consists” can be used interchangeably.

“Treatment,” “treating,” “palliating” and “ameliorating” (and grammatical variants of these terms), as used herein, are used interchangeably. These terms refer to an approach for obtaining beneficial or desired results including but not limited to therapeutic benefit. A therapeutic benefit is achieved with the eradication or amelioration of one or more of the physiological symptoms associated with BC such that an improvement is observed in the patient, notwithstanding that the patient may still be afflicted with BC.

“Subject” refers to an animal, such as a mammal, for example a human. The methods described herein can be useful in both humans and non-human animals. In some embodiments, the subject is a mammal (such as an animal model of disease), and in some embodiments, the subject is human.

All patents, patent applications, provisional applications, and publications referred to or cited herein are incorporated by reference in their entirety, including all figures and tables, to the extent they are not inconsistent with the explicit teachings of this specification.

### Materials and Methods

### Clinical Sampling and Processing

Urine samples and associated clinical information were consecutively collected from subjects visiting the urology clinic. The discovery cohort consisted of 58 individuals with no evidence of active urothelial cell carcinoma (controls) and 27 individuals with primary urothelial carcinoma (cases). The validation cohort consisted of 60 individuals with no evidence of active urothelial cell carcinoma (controls) and 61 individuals with newly diagnosed primary urothelial carcinoma (cases).

All subjects underwent standard clinical work-up, including office cystoscopy. A majority of subjects also had axial imaging of the abdomen and pelvis. For the BC group, histological confirmation of urothelial carcinoma, including grade and stage was defined from excised tissue.

A summary of clinical data for both cohorts is given in Table 1.

TABLE 1
Demographic and clinicopathologic characteristics of study cohorts

| | Cohort 1 | | | | Cohort 2 | | | |
|---|---|---|---|---|---|---|---|---|
| | Controls | | Cases | | Controls | | Cases | |
| | N = 58 | | N = 27 | | N = 60 | | N = 61 | |
| Median | 61 | (20-88) | 66 | (52-87) | 60.5 | (19-90) | 70 | (29-94) |
| Age | | | | | | | | |
| (range, | | | | | | | | |
| years) | | | | | | | | |
| Gender | | | | | | | | |
| Male | 47 | (82.5%) | 19 | (70.4%) | 47 | (79.7%) | 50 | (86.2%) |
| Female | 10 | (17.5%) | 7 | (25.9%) | 12 | (20.3%) | 8 | (13.8%) |
| Missing | 1 | | 1 | | 1 | | 3 | |
| Race | | | | | | | | |
| White | 38 | (65.5%) | 23 | (85.2%) | 42 | (70.0%) | 44 | (72.1%) |
| African | 7 | (12.1%) | 1 | (3.7%) | 3 | (3.0%) | 5 | (8.2%) |
| American | | | | | | | | |
| Other | 4 | (6.90%) | 1 | (3.7%) | 4 | (6.7%) | 5 | (8.2%) |
| Unknown | 9 | | 2 | | 11 | | 7 | |
| Clinical | | | | | | | | |
| stage | | | | | | | | |
| Ta | n/a | | 4 | (14.8%) | n/a | | 15 | (27.8%) |
| Cis | n/a | | 3 | (11.1%) | n/a | | 6 | (11.1%) |
| T1 | n/a | | 9 | (33.3%) | n/a | | 13 | (24.1%) |
| T2 | n/a | | 7 | (25.9%) | n/a | | 15 | (27.8%) |
| T3 | n/a | | 3 | (11.1%) | n/a | | 5 | (9.3%) |
| Missing | | | 1 | | | | 7 | |
| Grade | | | | | | | | |
| High | n/a | | 22 | (81.5%) | n/a | | 41 | (83.7%) |
| Low | n/a | | 1 | (3.7%) | n/a | | 8 | (16.3%) |
| Missing | n/a | | 4 | | n/a | | 12 | |
| Hematuria | | | | | | | | |
| No | 51 | (94.4%) | 19 | (70.4%) | 49 | (90.7%) | 45 | (77.6%) |
| Yes | 3 | (5.6%) | 6 | (22.2%) | 5 | (9.3%) | 13 | (22.4%) |
| Missing | 4 | | 2 | | 6 | | 3 | |
| Cytology | | | | | | | | |
| results | | | | | | | | |
| Negative | n/a | | 11 | (40.7%) | n/a | | 20 | (42.6%) |
| Positive | n/a | | 8 | (29.6%) | n/a | | 22 | (46.8%) |
| Reactive | n/a | | 2 | (7.4%) | n/a | | 3 | (6.4%) |
| Suspicious | n/a | | 2 | (7.4%) | n/a | | 2 | (4.3%) |
| Missing | | | 4 | | 0 | | 14 | |

Prior to any intrusive investigation or treatment, 30-50 ml of midstream voided urine was collected from each subject in a sterile cup and stored at 4° C. until processing, preferably within 3 hours of sample collection.

Each sample was assigned a unique identifying number before laboratory processing. Urothelial cells were pelleted from the total urine sample by centrifugation (600 g at 4° C. for 5 min), rinsed in PBS, pelleted again, and frozen for storage at −80 C. Total RNA was purified using Qiagen RNeasy kit with subsequent Qiagen DNase treatment. RNA samples were evaluated quantitatively and qualitatively using an Agilent Bioanalyzer 2000, before storage at −80° C. as previously described.

### Quantitative Real-Time PCR Analysis

Profiling of 754 human miRNAs was performed using TaqMan® Array Human MicroRNA A+B Cards Set v3.0 (Applied Biosystems Cat #4444913). The TLDA format is a 384-well system that uses standard TaqMan® assays and enables automated loading and high-throughput analyses. Details on the included assays are available in the Target List file (Applied Biosystems website), and each array included an endogenous control (Mamm U6) for data normalization. Megaplex™ RT Primers, Human Pool Set v3.0 and Megaplex™ PreAmp Primers (Applied Biosystems) were used for cDNA synthesis and preamplification respectively. Custom TLDAs for the validation studies were constructed by Applied Biosystems (AB) upon request. Targets included Mamm U6 as endogenous control plus 25 miRNAs biomarkers identified as associated with the presence of BC from the discovery profiling analysis. Targets for validation were selected by statistical ranking (p-value) and fold-change, including 4 targets negatively associated with BC. The complete list of targets selected for validation is provided in Table 2.

TABLE 2
miRNA profiling RT-PCR data. Profiling (754 targets) was performed
on an 85-subject cohort (cohort 1). Differential expression
data for miRNA targets selected for validation are shown.

| | | t-test |
|---|---|---|
| miRNA | Fold Change | P value |
| hsa-miR-140-5p | 18.06 | <0.0001 |
| hsa-miR-142-5p | 8.25 | <0.0001 |
| hsa-miR-199a-3p | 14.11 | <0.0001 |
| hsa-miR-93-5p | 18.33 | <0.0001 |
| hsa-miR-652-3p | 14.30 | <0.0001 |
| hsa-miR-106b-3p | 7.28 | <0.0001 |
| hsa-miR-1305 | −108.00 | <0.0001 |
| hsa-miR-223-3p | 10.58 | <0.0001 |
| hsa-miR-191-5p | 6.24 | <0.0001 |
| hsa-miR-30a-3p | −2.80 | 0.0014 |
| hsa-miR-96-5p | 2.63 | 0.0166 |
| hsa-miR-224-5p | 4.97 | 0.0008 |
| hsa-miR-766-3p | 2.30 | 0.0282 |
| hsa-miR-146b-5p | 11.14 | <0.0001 |
| hsa-miR-429 | 3.70 | 0.0005 |
| hsa-miR-200a-3p | 8.46 | <0.0001 |
| hsa-miR-143-3p | 6.41 | 0.0003 |
| hsa-miR-99b-5p | 9.82 | <0.0001 |
| hsa-miR-140-3p | 4.50 | <0.0001 |
| hsa-miR-491-5p | 4.03 | 0.0002 |
| hsa-miR-222-3p | 2.91 | 0.0054 |
| hsa-miR-339-3p | 3.81 | 0.0002 |
| hsa-miR-141-3p | 2.56 | 0.0127 |
| hsa-miR-200b-3p | 6.94 | 0.0031 |
| hsa-let-7b-5p | 8.43 | 0.0006 |

DDCt = DCt (cases) − DCt (controls)
Fold Change (cases/controls) = 2^{-DDCT}
Negative FC values indicate target is down-regulated in BC cases
Table 2 contains latest miRNA identifiers according to miRNA database miRBase version 21, available at world-wide-website: mirbase.org

The PCR reactions were run on a 7900HT Fast Real-Time PCR System (Applied Biosystems). RT-PCR amplification results were processed with RQ manager (Applied Biosystems). The baseline correction was manually checked for each target and the Ct threshold was set to 0.2 for every target across all plates. Samples used for downstream analysis were required to be positive for control genes. Targets deemed to be undetermined (Ct>40) were given a Ct 40 value.

### Statistical Analysis

For the 754-target profiling analyses, Delta Ct (DCt) values were calculated by normalization with the endogenous reference Mamm U6 miRNA and the fold-change between BC and control samples was calculated as log 2-DDCT. Differential miRNA expression was analyzed using a t-test comparison between the mean DCt values of BC cases and controls. For the validation study analyses (cohort 2), differences in clinical covariates between BC cases and non-malignant controls were evaluated via Chi-squared test and Wilcoxon Rank Sum test, as appropriate. For each miRNA, the percentage of samples that were censored (Ct value=40) was calculated for cases and controls separately (Table 2). To avoid biased inference caused by the issue of RT-PCR non-detects (Ct value=40), a left-censoring approach was used. Ct values of 40 were substituted with the highest observed Ct value for a given miRNA. Ct values were then normalized by subtracting the Ct value of the endogenous control (Mamm U6) from each of the 25 miRNAs of interest. For each miRNA, left-censored Tobit models were used to test for differences in miRNA expression between cases and controls. Multivariable logistic models were used to develop a signature to predict BC diagnosis. All miRNAs with less than 50% censoring were considered in the multivariable models used to shrink the model coefficients. ROC curves and associated AUCs were calculated to assess the performance of the multivariable models. The sensitivity and specificity associated with the maximum Youden index was selected from each ROC curve. Left-censored Tobit models were additionally used to evaluate associations between miRNA expression and clinical variables. Results with P<0.05 were deemed statistically significant.

Following are examples that illustrate procedures for practicing the invention. These examples should not be construed as limiting. All percentages are by weight and all solvent mixture proportions are by volume unless otherwise noted.

### Example 1—Urothelial Cell miRNA Profiling

A panel of 754 human miRNAs was monitored in a set of urothelial samples obtained from a total of 85 subjects of known bladder disease status, 27 of which had biopsy-proven BC (cohort 1). Of the 754 miRNAs included on the TaqMan® low density arrays (Human MicroRNA Set v3.0), 267 were detected in urothelial cell samples. Comparative group (cases vs. controls) analysis identified 108 miRNAs that were significantly associated (p<0.05) with BC. A set of 25 miRNAs from the broad-spectrum, discovery profiling (Table 2) was selected for validation in an independent cohort.

### Example 2—Association of Candidate miRNA Biomarkers with BC

The candidate miRNA biomarkers were tested in urine samples obtained from an independent cohort of 121 subjects, 61 with confirmed BC (Table 2). While RT-PCR analysis confirmed that the control miRNA was detected in all samples tested, the expression of the candidate miRNA markers was more variable.

Univariate Tobit model results for testing the association of 25 candidate miRNA biomarkers with case-control status. Biomarkers are ranked by Tobit model p-value. Because of censoring, the Tobit model estimate represents the difference between cases and controls in the un-observed latent variable.

To avoid bias introduced by the issue of RT-PCR non-detects, a left-censoring statistical approach to determine per-target differential expression in cases versus controls. Table 2 provides univariate differential expression results for each biomarker. Additional information on biomarker candidacy was obtained by evaluating the association with specific clinical factors or distinct subsets of patients. Such identified associations could impact decisions regarding inclusion in a test panel for a specific clinical utility. Left-censored Tobit models were used to estimate and compare associations of biomarkers with clinical factors (hematuria, tumor grade, clinical stage, age, sex). Very few of the top-ranked candidate biomarkers (Tobit model p<0.05) were significantly associated with gender, age or hematuria (four, three and zero, respectively). Four miRNAs were significantly associated with tumor grade, and three with muscle-invasive disease. Notably, miR-199a-3p was associated with grade, invasive disease, age, and sex.

### Example 3—Multivariate Analysis and Prediction Modeling

Multivariate logistic models were constructed to identify multifactorial gene sets that could predict the case-control status of a given sample. The LASSO approach was used to shrink model coefficients and model performance was described using receiver operating characteristic (ROC) analysis. Corresponding odds ratios for the multivariate logistic regression models are shown in Table 3.

Table 3. Multivariate logistic diagnostic models. The Lasso method was used to shrink model coefficients. The corresponding odds ratios are provided for models comprised of 25, 20, 15 and 10 miRNAs.

TABLE 3
Multivariate logistic diagnostic models.

| | 25- | 20- | 15- | 10- |
|---|---|---|---|---|
| | miRNA | miRNA | miRNA | miRNA |
| miRNA | model | model | model | model |
| hsa-miR-652-3p | 1.137 | 1.088 | 1.061 | 1.065 |
| hsa-miR-199a-3p | 1.313 | 1.255 | 1.186 | 1.146 |
| hsa-miR-140-5p | 1.093 | 1.107 | 1.129 | 1.092 |
| hsa-miR-93-5p | 1.462 | 1.243 | 1.113 | 1.119 |
| hsa-miR-142-5p | 1.048 | 1.043 | 1.043 | 1.031 |
| hsa-miR-1305 | 0.807 | 0.862 | 0.894 | 0.921 |
| hsa-miR-30a-3p | 0.907 | 0.88 | 0.87 | 0.946 |
| hsa-miR-224-5p | 1.203 | 1.114 | 1.054 | 1.008 |
| hsa-miR-96-5p | 1.109 | 1.084 | 1.048 | 1.02 |
| hsa-miR-766-3p | 0.755 | 0.794 | 0.825 | 0.865 |
| hsa-miR-223-3p | 1.135 | 1.037 | 1.024 | |
| hsa-miR-99b-5p | 1.468 | 1.278 | 1.15 | |
| hsa-miR-140-3p | 0.881 | 0.935 | 0.99 | |
| hsa-let-7b-5p | 0.556 | 0.774 | 0.968 | |
| hsa-miR-141-3p | 0.677 | 0.833 | 0.998 | |
| hsa-miR-191-5p | 1.694 | 1.129 | | |
| hsa-miR-146b-5p | 0.854 | 0.988 | | |
| hsa-miR-491-5p | 0.86 | 0.994 | | |
| hsa-miR-339-3p | 0.892 | 0.987 | | |
| hsa-miR-200c-3p | 1.497 | 1.215 | | |
| hsa-miR-106b-3p | 1.054 | | | |
| hsa-miR-143-3p | 0.973 | | | |
| hsa-miR-429 | 1.12 | | | |
| hsa-miR-222-3p | 0.999 | | | |
| hsa-miR-200a-3p | 1.003 | | | |

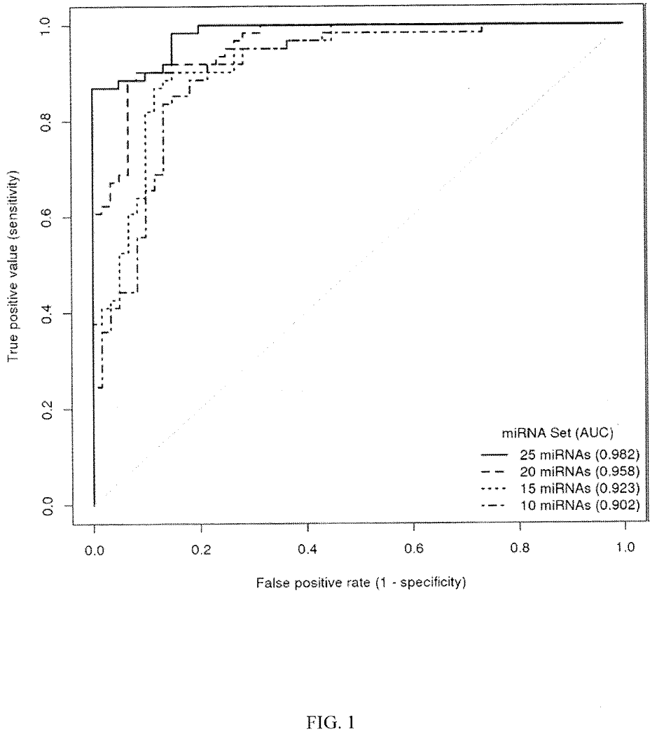

A 25-miRNA prediction model derived from a combination of all candidate biomarkers achieved optimal performance; AUC 0.982, sensitivity of 87% and specificity of 100% (FIG. 1). It is of interest to assess the adjustment of model performance when a limited number, for example, 10, 15, 20, of the candidate biomarkers are included. This may aid in the development of less complex assays for future clinical adoption, and can reveal which miRNAs comprise the core of the predictive models. Restriction to 20 miRNAs identified a predictive model (FIG. 1) with a AUC of 0.958, a 15-miRNA model achieved AUC of 0.923, and a 10-miRNA model achieved an AUC of 0.902. Of the cases in this cohort that had VUC data available, VUC evaluation positively identified 47%.

### Example 4—Identification of miRNA Panels Associated with BC

The development of accurate, non-invasive urinary assays for BC is provided. The assays facilitate the detection and management of BC, which has a high rate of recurrence and progression. To identify miRNA signatures with potential for non-invasive diagnosis, a discovery and validation strategy was employed using urothelial cell samples that are naturally shed from the bladder lining and can be readily recovered from urine. The rationale for analyzing the shed urothelial component of urine is two-fold. Firstly, the analysis of the component that will be the analyte of a future assay is optimal. Secondly, the analyte enables comparison of samples collected from subjects with non-malignant conditions. Conversely, truly normal bladder tissues are rarely available from surgically excised material.

The profiling of 754 miRNAs in one set of samples enabled the selection of 25 BC-associated targets for quantitative validation in an independent cohort using a custom-designed TLDA. Analysis of the validation cohort confirmed that the majority of the candidate miRNA biomarkers were associated with BC; however, association with specific clinical variables was much less evident.

In the assays provided by the instant invention, the optimal miRNA signature comprised of up to 25 targets, and with improving quantitative PCR technologies the monitoring of multiplex RNA panels is not a limiting factor. The limiting of prediction models to 15 and 10 miRNAs expectedly resulted in some loss of performance; however, it also revealed several miRNAs (miR-140-5p, miR-199a-3p, miR-93, miR-652, miR-1305, miR-224, miR-96, miR-766) that consistently contributed to all models. The majority of these core miRNAs have not been reported to be associated with BC.

### REFERENCES

The following references are incorporated by reference in their entirety, including all figures and tables, to the extent they are not inconsistent with the explicit teachings of this specification.
1. Chou R, Gore J L, Buckley D, Fu R, Gustafson K, Griffin J C, Grusing S and Selph S. Urinary Biomarkers for Diagnosis of Bladder Cancer: A Systematic Review and Meta-analysis. Annals of internal medicine. 2015; 163(12):922-931.
2. Dyrskjot L, Ostenfeld M S, Bramsen J B, Silahtaroglu A N, Lamy P, Ramanathan R, Fristrup N, Jensen J L, Andersen C L, Zieger K, Kauppinen S, Ulhoi B P, Kjems J, Borre M and Orntoft T F. Genomic profiling of microRNAs in bladder cancer: miR-129 is associated with poor outcome and promotes cell death in vitro. Cancer research. 2009; 69(11):4851-4860.
3. Iorio M V, Ferracin M, Liu C G, Veronese A, Spizzo R, Sabbioni S, Magri E, Pedriali M, Fabbri M, Campiglio M, Menard S, Palazzo J P, Rosenberg A, Musiani P, Volinia S, Nenci I, et al. MicroRNA gene expression deregulation in human breast cancer. Cancer research. 2005; 65(16):7065-7070.
4. Han Y, Chen J, Zhao X, Liang C, Wang Y, Sun L, Jiang Z, Zhang Z, Yang R, Chen J, Li Z, Tang A, Li X, Ye J, Guan Z, Gui Y, et al. MicroRNA expression signatures of bladder cancer revealed by deep sequencing. PloS one. 2011; 6(3):e18286.
5. Urquidi V, Netherton M, Gomes-Giacoia E, Serie D J, Eckel-Passow J, Rosser C J, Goodison S A microRNA biomarker panel for the non-invasive detection of bladder cancer. Oncotarget. 2016 Dec. 27; 7(52):86290-86299

We claim:

1. A method for treating bladder cancer (BC) in a subject, the method comprising the steps of:
(a) determining the level of two or more microRNAs (miRNAs) in:
i) a test sample obtained from the subject, and
ii) optionally a control sample;
(b) optionally obtaining two or more reference values corresponding to levels of one or miRNAs, and
(c) identifying the subject as having BC based on the level of two or more miRNAs in the test sample and optionally, administering a therapy to the subject to treat BC, or
(d) identifying the subject as not having BC based on the level of two or more miRNAs in the test sample and withholding the therapy to the subject to treat BC;
wherein the two or more miRNAs are selected from the miRNAs in Panel 25.

2. The method of claim 1, wherein administering the therapy to the subject to treat and/or manage BC comprises administering to the subject a pharmaceutically effective amount of: i) one or more antisense miRNAs that target one or more miRNAs from Panel 25 that are upregulated in BC, ii) one or more miRNAs that are downregulated in BC, or a combination of i) and ii).

3. The method of claim 1, wherein the control sample and the test sample are obtained from the same type of a body fluid, and wherein the body fluid is urine, blood, serum, or plasma.

4. The method of claim 1, wherein the levels of miRNAs in the test sample and optionally, the control sample, are determined by microarray analysis, real-time polymerase chain reaction (PCR), Northern blot, in situ hybridization, solution hybridization, or quantitative reverse transcription PCR (qRT-PCR).

5. The method of claim 1, wherein the subject is a human.

6. The method of claim 5, wherein the combination of the two or more miRNAs in Panel 25, consists essentially of the miRNAs in: Panel 10, Panel 15, Panel 20, or Panel 25.

7. The method of claim 6, wherein the combination of the two or more miRNAs permit identifying BC with specificity of at least 80% and sensitivity of at least 80%.

8. The method of claim 5, wherein treating BC in the subject comprises administering to the subject: a) one or more antisense miRNAs that target one or more miRNAs from Panel 25 that are upregulated in BC, b) one or more miRNAs from Panel 25 that are downregulated in BC, or a combination of a) and b).

9. An oligonucleotide chip consisting essentially of oligonucleotides corresponding to two or more miRNAs selected from the miRNAs in Panel 25, wherein the combination of the two or more miRNAs permit identifying BC with specificity of at least 80% and sensitivity of at least 80% in a subject.

10. The oligonucleotide chip of claim 9, consisting essentially of the oligonucleotides corresponding to the miRNAs in: Panel 10, Panel 15, Panel 20, or Panel 25.

11. An assay for determining the levels of a minimum of three and a maximum of twenty-five miRNAs in:
i) a test sample obtained from the subject, and
ii) optionally, a control sample;
wherein each of the assayed minimum of three and the maximum of twenty-five miRNAs is selected from the miRNAs in Panel 25, wherein the Panel 25 consists of the following miRNAs: hsa-miR-652-3p, hsa-miR-199a-3p, hsa-miR-140-5p, hsa-miR-93-5p, hsa-miR-142-5p, hsa-miR-1305, hsa-miR-30a-3p, hsa-miR-224-5p, hsa-miR-96-5p, hsa-miR-766-3p, hsa-miR-223-3p, hsa-miR-99b-5p, hsa-miR-140-3p, hsa-let-7b-5p, hsa-miR-141-3p, hsa-miR-191-5p, hsa-miR-146b-5p, hsa-miR-491-5p, hsa-miR-339-3p, hsa-miR-200c-3p, hsa-miR-106b-3p, hsa-miR-143-3p, hsa-miR-429, hsa-miR-222-3p, and hsa-miR-200a-3p.

12. The assay of claim 11, wherein the step of determining the levels of the minimum of three and the maximum of twenty-five miRNAs in the test sample, and if obtained, the control sample, comprises conducting a microarray analysis, real-time polymerase chain reaction (PCR), Northern blot, in situ hybridization, solution hybridization, or quantitative reverse transcription PCR (qRT-PCR).

13. The assay of claim 11, comprising determining in the test sample and, if obtained, the control sample, the levels of the minimum of three and the maximum of twenty-five miRNAs selected from the miRNAs in the Panel 25,
wherein the assay comprises:
a) contacting the test sample, and if obtained, the control sample, with a minimum of three and a maximum of twenty-five oligonucleotides, wherein each oligonucleotide from the minimum of three and the maximum of twenty-five oligonucleotides hybridizes with one miRNA from the assayed minimum of three and the maximum of twenty-five miRNAs in the Panel 25, and
c) detecting the presence and quantity of the oligonucleotide-miRNA complexes that form in the test sample, and if obtained, the control sample.

14. The assay of claim 13, comprising determining the level of each miRNA in Panel 10, Panel 15, Panel 20, or Panel 25, wherein:
i) Panel 10 consists of the following miRNAs: hsa-miR-652-3p, hsa-miR-199a-3p, hsa-miR-140-5p, hsa-miR-93-5p, hsa-miR-142-5p, hsa-miR-1305, hsa-miR-30a-3p, hsa-miR-224-5p, hsa-miR-96-5p, and hsa-miR-766-3p;
ii) Panel 15 consists of the following miRNAs: hsa-miR-652-3p, hsa-miR-199a-3p, hsa-miR-140-5p, hsa-miR-93-5p, hsa-miR-142-5p, hsa-miR-1305, hsa-miR-30a-3p, hsa-miR-224-5p, hsa-miR-96-5p, hsa-miR-766-3p, hsa-miR-223-3p, hsa-miR-99b-5p, hsa-miR-140-3p, hsa-let-7b-5p, and hsa-miR-141-3p;
iii) Panel 20 consists of the following miRNAs: hsa-miR-652-3p, hsa-miR-199a-3p, hsa-miR-140-5p, hsa-miR-93-5p, hsa-miR-142-5p, hsa-miR-1305, hsa-miR-30a-3p, hsa-miR-224-5p, hsa-miR-96-5p, hsa-miR-766-3p, hsa-miR-223-3p, hsa-miR-99b-5p, hsa-miR-140-3p, hsa-let-7b-5p, hsa-miR-141-3p, hsa-miR-191-5p, hsa-miR-146b-5p, hsa-miR-491-5p, hsa-miR-339-3p, and hsa-miR-200c-3p; and
iv) Panel 25 consists of the following miRNAs: hsa-miR-652-3p, hsa-miR-199a-3p, hsa-miR-140-5p, hsa-miR-93-5p, hsa-miR-142-5p, hsa-miR-1305, hsa-miR-30a-3p, hsa-miR-224-5p, hsa-miR-96-5p, hsa-miR-766-3p, hsa-miR-223-3p, hsa-miR-99b-5p, hsa-miR-140-3p, hsa-let-7b-5p, hsa-miR-141-3p, hsa-miR-191-5p, hsa-miR-146b-5p, hsa-miR-491-5p, hsa-miR-339-3p, hsa-miR-200c-3p, hsa-miR-106b-3p, hsa-miR-143-3p, hsa-miR-429, hsa-miR-222-3p, and hsa-miR-200a-3p.

15. The assay of claim 11, comprising determining in the test sample and, if obtained, the control sample, the levels of the minimum of three and the maximum of twenty-five miRNAs selected from the miRNAs in the Panel 25,
wherein the assay comprises:
a) contacting the test sample, and if obtained, the control sample, with a minimum of three and a maximum of twenty-five combinations of primer pairs and probes and conducting a PCR, wherein each combination of a primer pair and a probe from said minimum of three and a maximum of twenty-five combinations of primer pairs and probes amplifies one miRNA from the assayed minimum of three and the maximum of twenty-five miRNAs in the Panel 25, and
c) detecting the presence and quantity of the minimum of three and the maximum of twenty-five miRNAs based on the qPCR in the test sample, and if obtained, the control sample.

16. The assay of claim 15, comprising determining the levels of miRNAs consisting of the miRNAs in the Panel 10, Panel 15, Panel 20, or Panel 25.

17. The assay of claim 11, wherein the test sample is obtained from a human subject and the control sample is obtained from: a human not having bladder cancer (BC) or the human subject at a time the human subject was known to be free from BC.

18. The assay of claim 17, wherein the control sample and the test sample are obtained from a same type of body fluid.

19. The assay of claim 18, wherein the body fluid is urine, blood, serum or plasma.

20. The assay of claim 11, comprising determining the levels of a minimum of five and a maximum of twenty-five miRNAs.


SEQUENCE LISTING (92 sequences)

SEQ ID NO: 1 - RNA, 22 nt, Homo sapiens
cagugguuuuacccuaugguag

SEQ ID NO: 2 - RNA, 100 nt, Homo sapiens
ugugucucucucuguguccugccagugguuuuacccuaugguagguuacgucaugcuguucuaccacaggguagaaccacggacaggauaccggggcacc

SEQ ID NO: 3 - RNA, 21 nt, Homo sapiens
cauaaaguagaaagcacuacu

SEQ ID NO: 4 - RNA, 87 nt, Homo sapiens
gacagugcagucacccauaaaguagaaagcacuacuaacagcacuggaggguguaguguuuccuacuuuauggaugaguguacugug

SEQ ID NO: 5 - RNA, 22 nt, Homo sapiens
acaguagucugcacauugguua

SEQ ID NO: 6 - RNA, 71 nt, Homo sapiens
gccaacccaguguucagacuaccuguucaggaggcucucaauguguacaguagucugcacauugguuaggc

SEQ ID NO: 7 - RNA, 23 nt, Homo sapiens
caaagugcuguucgugcagguag

SEQ ID NO: 8 - RNA, 80 nt, Homo sapiens
cugggggcuccaaagugcuguucgugcagguagugugauuaccugaccuacugcugagcuagcacuucccgagcccccgg

SEQ ID NO: 9 - RNA, 21 nt, Homo sapiens
aauggcgccacuaggguugug

SEQ ID NO: 10 - RNA, 98 nt, Homo sapiens
acgaauggcuaugcacugcacaacccuaggagagggugccauucacauagacuauaauugaauggcgccacuaggguugugcagugcacaaccuacac

SEQ ID NO: 11 - RNA, 23 nt, Homo sapiens
uaaagugcuuauagugcagguag

SEQ ID NO: 12 - RNA, 71 nt, Homo sapiens
guagcacuaaagugcuuauagugcagguaguguuuaguuaucuacugcauuaugagcacuuaaaguacugc

SEQ ID NO: 13 - RNA, 22 nt, Homo sapiens
ccgcacuguggguacuugcugc

SEQ ID NO: 14 - RNA, 82 nt, Homo sapiens
ccugccggggcuaaagugcugacagugcagauagugguccucuccgugcuaccgcacuguggguacuugcugcuccagcagg

SEQ ID NO: 15 - RNA, 22 nt, Homo sapiens
uuuucaacucuaaugggagaga

SEQ ID NO: 16 - RNA, 86 nt, Homo sapiens
aagauccugcuguuucuaccauuaguuuugaauguuuauuguaaagauacuuuucaacucuaaugggagagacagcaggauucucc

SEQ ID NO: 17 - RNA, 22 nt, Homo sapiens
ugucaguuugucaaauacccca

SEQ ID NO: 18 - RNA, 110 nt, Homo sapiens
ccuggccuccugcagugccacgcuccguguauuugacaagcugaguuggacacuccaugugguagagugucaguuugucaaauaccccaagugcggcacaugcuuaccag

SEQ ID NO: 19 - RNA, 23 nt, Homo sapiens
uaaggugcaucuagugcagauag

SEQ ID NO: 20 - RNA, 71 nt, Homo sapiens
uguucuaaggugcaucuagugcagauagugaaguagauuagcaucuacugcccuaagugcuccuucuggca

SEQ ID NO: 21 - RNA, 23 nt, Homo sapiens
caacggaaucccaaaagcagcug

SEQ ID NO: 22 - RNA, 92 nt, Homo sapiens
cggcuggacagcgggcaacggaaucccaaaagcagcuguugucuccagagcauuccagcugcgcuuggauuucguccccugcucuccugccu

SEQ ID NO: 23 - RNA, 22 nt, Homo sapiens
ucguaccgugaguaauaaugcg

SEQ ID NO: 24 - RNA, 85 nt, Homo sapiens
cgcuggcgacgggacauuauuacuuuugguacgcgcugugacacuucaaacucguaccgugaguaauaaugcgccguccacggca

SEQ ID NO: 25 - RNA, 21 nt, Homo sapiens
uucaaguaauucaggauaggu

SEQ ID NO: 26 - RNA, 76 nt, Homo sapiens
ccgggacccaguucaaguaauucaggauaggugugugcuguccagccuguucuccauuacuuggcucggggaccgg

SEQ ID NO: 27 - RNA, 22 nt, Homo sapiens
uucaaguaauccaggauaggcu

SEQ ID NO: 28 - RNA, 77 nt, Homo sapiens
guggccucguucaaguaauccaggauaggcugugcaggucccaaugggccuauucuugguuacuugcacggggacgc

SEQ ID NO: 29 - RNA, 23 nt, Homo sapiens
guccaguuuucccaggaaucccu

SEQ ID NO: 30 - RNA, 88 nt, Homo sapiens
caccuuguccucacgguccaguuuucccaggaaucccuuagaugcuaagauggggauuccuggaaauacuguucuugaggucaugguu

SEQ ID NO: 31 - RNA, 22 nt, Homo sapiens
ugagaacugaauuccauggguu

SEQ ID NO: 32 - RNA, 99 nt, Homo sapiens
ccgauguguauccucagcuuugagaacugaauuccauggguugugucagugucagaccucugaaauucaguucuucagcugggauaucucugucaucgu

SEQ ID NO: 33 - RNA, 22 nt, Homo sapiens
cuuucagucggauguuugcagc

SEQ ID NO: 34 - RNA, 71 nt, Homo sapiens
gcgacuguaaacauccucgacuggaagcugugaagccacagaugggcuuucagucggauguuugcagcugc

SEQ ID NO: 35 - RNA, 23 nt, Homo sapiens
uuuggcacuagcacauuuuugcu

SEQ ID NO: 36 - RNA, 78 nt, Homo sapiens
uggccgauuuuggcacuagcacauuuuugcuugugucucuccgcucugagcaaucaugugcagugccaauaugggaaa

SEQ ID NO: 37 - RNA, 24 nt, Homo sapiens
cugaagugauguguaacugaucag

SEQ ID NO: 38 - RNA, 99 nt, Homo sapiens
uuuagcgguuucucccugaagugauguguaacugaucaggaucuacucaugucgucuuugguaaaguuaugucgcuugucagggugaggagaguuuuug

SEQ ID NO: 39 - RNA, 23 nt, Homo sapiens
agcuacauugucugcuggguuuc

SEQ ID NO: 40 - RNA, 110 nt, Homo sapiens
ugaacauccaggucuggggcaugaaccuggcauacaauguagauuucuguguucguuaggcaacagcuacauugucugcuggguuucaggcuaccuggaaacauguucuc

SEQ ID NO: 41 - RNA, 24 nt, Homo sapiens
uuuggcaaugguagaacucacacu

SEQ ID NO: 42 - RNA, 110 nt, Homo sapiens
gagcugcuugccuccccccguuuuuggcaaugguagaacucacacuggugagguaacaggauccggugguucuagacuugccaacuauggggcgaggacucagccggcac

SEQ ID NO: 43 - RNA, 23 nt, Homo sapiens
uguaguguuuccuacuuuaugga

SEQ ID NO: 44 - RNA, 87 nt, Homo sapiens; identical to SEQ ID NO: 4

SEQ ID NO: 45 - RNA, 23 nt, Homo sapiens
ugugcaaauccaugcaaaacuga

SEQ ID NO: 46 - RNA, 87 nt, Homo sapiens
cacuguucuaugguuaguuuugcagguuugcauccagcugugugauauucugcugugcaaauccaugcaaaacugacugugguagug

SEQ ID NO: 47 - RNA, 21 nt, Homo sapiens
caagucacuagugguuccguu

SEQ ID NO: 48 - RNA, 81 nt, Homo sapiens
gggcuuucaagucacuagugguuccguuuaguagaugauugugcauuguuucaaaauggugcccuagugacuacaaagccc

SEQ ID NO: 49 - RNA, 23 nt, Homo sapiens
aacauucaacgcugucggugagu

SEQ ID NO: 50 - RNA, 110 nt, Homo sapiens
agaagggcuaucaggccagccuucagaggacuccaaggaacauucaacgcugucggugaguuugggauuugaaaaaaccacugaccguugacuguaccuugggguccuua

SEQ ID NO: 51 - RNA, 22 nt, Homo sapiens
acuccagccccacagccucagc

SEQ ID NO: 52 - RNA, 111 nt, Homo sapiens
gcauccucaggaccugggcuugggugguaggaggaauuggugcuggucuuucauuuuggauuugacuccagccccacagccucagccaccccagccaauugucauaggagc

SEQ ID NO: 53 - RNA, 22 nt, Homo sapiens
ugagaacugaauuccauaggcu

SEQ ID NO: 54 - RNA, 73 nt, Homo sapiens
ccuggcacugagaacugaauuccauaggcugugagcucuagcaaugcccuguggacucaguucuggugcccgg

SEQ ID NO: 55 - RNA, 22 nt, Homo sapiens
uaauacugucugguaaaaccgu

SEQ ID NO: 56 - RNA, 83 nt, Homo sapiens
cgccggccgaugggcgucuuaccagacaugguuagaccuggcccucugucuaauacugucugguaaaaccguccauccgcugc

SEQ ID NO: 57 - RNA, 22 nt, Homo sapiens
uaacacugucugguaacgaugu

SEQ ID NO: 58 - RNA, 90 nt, Homo sapiens
ccgggccccugugagcaucuuaccggacagugcuggauuucccagcuugacucuaacacugucugguaacgauguucaaaggugacccgc

SEQ ID NO: 59 - RNA, 23 nt, Homo sapiens
uaauacugccggguaaugaugga

SEQ ID NO: 60 - RNA, 68 nt, Homo sapiens
cccucgucuuacccagcaguguuugggugcgguugggagucucuaauacugccggguaaugauggagg

SEQ ID NO: 61 - RNA, 23 nt, Homo sapiens
caaagugcucauagugcagguag

SEQ ID NO: 62 - RNA, 69 nt, Homo sapiens
aguaccaaagugcucauagugcagguaguuuuggcaugacucuacuguaguaugggcacuuccaguacu

SEQ ID NO: 63 - RNA, 20 nt, Homo sapiens
acugccccaggugcugcugg

SEQ ID NO: 64 - RNA, 83 nt, Homo sapiens
cugacuaugccuccccgcauccccuagggcauugguguaaagcuggagacccacugccccaggugcugcuggggguuguaguc

SEQ ID NO: 65 - RNA, 23 nt, Homo sapiens
ugugcaaaucuaugcaaaacuga

SEQ ID NO: 66 - RNA, 82 nt, Homo sapiens
gcaguccucuguuaguuuugcauaguugcacuacaagaagaauguaguugugcaaaucuaugcaaaacugaugguggccugc

SEQ ID NO: 67 - RNA, 23 nt, Homo sapiens
aaaagugcuuacagugcagguag

SEQ ID NO: 68 - RNA, 81 nt, Homo sapiens
ccuuggccauguaaaagugcuuacagugcagguagcuuuuugagaucuacugcaauguaagcacuucuuacauuaccaugg

SEQ ID NO: 69 - RNA, 21 nt, Homo sapiens
ugagaugaagcacuguagcuc

SEQ ID NO: 70 - RNA, 106 nt, Homo sapiens
gcgcagcgcccugucucccagccugaggugcagugcugcaucucuggucaguugggagucugagaugaagcacuguagcucaggaagagagaaguuguucugcagc

SEQ ID NO: 71 - RNA, 22 nt, Homo sapiens
cacccguagaaccgaccuugcg

SEQ ID NO: 72 - RNA, 70 nt, Homo sapiens
ggcacccacccguagaaccgaccuugcggggccuucgccgcacacaagcucgugucuguggguccguguc

SEQ ID NO: 73 - RNA, 21 nt, Homo sapiens
uaccacaggguagaaccacgg

SEQ ID NO: 74 - RNA, 100 nt, Homo sapiens; identical to SEQ ID NO: 2

SEQ ID NO: 75 - RNA, 22 nt, Homo sapiens
aguggggaacccuuccaugagg

SEQ ID NO: 76 - RNA, 84 nt, Homo sapiens
uugacuuagcuggguaguggggaacccuuccaugaggaguagaacacuccuuaugcaagauucccuucuaccuggcuggguugg

SEQ ID NO: 77 - RNA, 21 nt, Homo sapiens
cuagacugaagcuccuugagg

SEQ ID NO: 78 - RNA, 90 nt, Homo sapiens
uuuccugcccucgaggagcucacagucuaguaugucucauccccuacuagacugaagcuccuugaggacagggauggucauacucaccuc

SEQ ID NO: 79 - RNA, 21 nt, Homo sapiens
uccgguucucagggcuccacc

SEQ ID NO: 80 - RNA, 118 nt, Homo sapiens
gcaggugaacuggcaggccaggaagaggaggaagcccuggaggggcuggaggugauggauguuuuccuccgguucucagggcuccaccucuuucgggccguagagccagggcuggugc

SEQ ID NO: 81 - RNA, 21 nt, Homo sapiens
agcuacaucuggcuacugggu

SEQ ID NO: 82 - RNA, 110 nt, Homo sapiens
gcugcuggaagguguagguacccucaauggcucaguagccaguguagauccugucuuucguaaucagcagcuacaucuggcuacugggucucugauggcaucuucuagcu

SEQ ID NO: 83 - RNA, 23 nt, Homo sapiens
ugagcgccucgacgacagagccg

SEQ ID NO: 84 - RNA, 94 nt, Homo sapiens
cggggcggccgcucucccuguccuccaggagcucacgugugccugccugugagcgccucgacgacagagccggcgccugccccagugucugcgc

SEQ ID NO: 85 - RNA, 22 nt, Homo sapiens
uaacacugucugguaaagaugg

SEQ ID NO: 86 - RNA, 95 nt, Homo sapiens
cggccggcccuggguccaucuuccaguacaguguuggauggucuaauugugaagcuccuaacacugucugguaaagauggcucccggguggguuc

SEQ ID NO: 87 - RNA, 22 nt, Homo sapiens
uaauacugccugguaaugauga

SEQ ID NO: 88 - RNA, 95 nt, Homo sapiens
ccagcucgggcagccguggccaucuuacugggcagcauuggauggagucaggucucuaauacugccugguaaugaugacggcggagcccugcacg

SEQ ID NO: 89 - RNA, 22 nt, Homo sapiens
ugagguaguagguugugugguu

SEQ ID NO: 90 - RNA, 83 nt, Homo sapiens
cggggugagguaguagguugugugguuucagggcagugauguugccccucggaagauaacuauacaaccuacugccuucccug

SEQ ID NO: 91 - RNA, 22 nt, Homo sapiens
uagcuuaucagacugauguuga

SEQ ID NO: 92 - RNA, 72 nt, Homo sapiens
ugucggguagcuuaucagacugauguugacuguugaaucucauggcaacaccagucgaugggcugucugaca